(19) United States
(12) Patent Application Publication
(10) Pub. No.: US 2022/0339011 A1
(43) Pub. Date: Oct. 27, 2022

(54) FOUR-BAR LINKAGE ANKLE JOINT MECHANISM AND ANKLE PROSTHESIS SYSTEM

(71) Applicant: Synchro Motion, LLC, Franklin, TN (US)

(72) Inventors: Harrison Logan BARTLETT, Atlanta, GA (US); Brian Edward LAWSON, Ann Arbor, MI (US); Michael GOLDFARB, Franklin, TN (US)

(73) Assignee: Synchro Motion, LLC, Franklin, TN (US)

(21) Appl. No.: 17/763,605

(22) PCT Filed: Sep. 24, 2020
(86) PCT No.: PCT/US2020/052450

§ 371 Date: Mar. 24, 2022

Related U.S. Application Data
(60) Provisional application No. 62/905,796, filed on Sep. 25, 2019.

(51) Int. Cl. A61F 2/66; A61F 2/74

(52) U.S. Cl. CPC A61F 2/6607; A61F 2/74; A61F 2002/5038

(57) ABSTRACT

The present disclosure provides an ankle joint mechanism, including a shank member, a first connecting link, a foot member, a second connecting link, and a force providing element. The foot member is coupled to the shank member at a first pivot point and coupled to the first connecting link at a second pivot point. The second connecting link is coupled to the first connecting link at a third pivot point and coupled to the shank member at a fourth pivot point. The force providing element is coupled to the second connecting link at a first end and coupled to either the shank member or the foot member at a second end.

This filing claims priority to under 35 U.S.C. § 119 to U.S. Provisional Application No. 62/905,796, entitled “A Single-Axis Four Bar Mechanism for a Prosthetic Ankle Joint,” filed on Sep. 25, 2019, the contents of which are hereby incorporated by reference in their entirety.

### FIELD OF THE DISCLOSURE

This disclosure relates to a four-bar linkage mechanism as used in a joint and/or an ankle prosthesis or orthosis.

### BACKGROUND

Conventional prosthetic ankle joints are typically non-articulated (i.e., not jointed) and consist of a spring-like material, which is statically configured at a nominal ankle angle and deflects when loaded. Accordingly, unlike the anatomical ankle joint, these conventional prosthetic devices do not have a well-defined axis of rotation between the shank and foot segments. A conventional prosthetic device presents grave challenges to users when navigating sloping and uneven terrain or varying shoe geometry (i.e. shoes with varying heel-heights), as a result of the prosthesis's (1) static, spring-like design, (2) lack of articulation, and (3) limited range of motion.

Some conventional prosthetic ankle joints are articulated with a rotary joint connecting the shank and foot segments. In some examples, these articulated ankle devices utilize actuators acting between the foot and shank segments to provide the dynamic behavior of the ankle/foot complex. These actuators may be energetically passive or active and may be controlled by a microprocessor to provide dynamic behavior. However, these actuators are typically large and heavy in order to provide appropriate biomechanical functionality. Reducing the size and weight of actuators in conventionally-designed articulated prosthetic ankle joints trades off by reducing the biomechanical functionality.

Although conventional prosthetic ankle joints are typically non-articulated, prosthetic joints for other human joints are articulated (e.g., elbow or knee prostheses). Elbow or knee prosthesis joints (e.g., the prosthetic knee joint shown in DE102014015756B3) cannot be translated for use in an ankle joint because of sizing constraints and loading constraints of the materials to prevent buckling.

### SUMMARY

The following presents a simplified summary in order to provide a basic understanding of some aspects described herein. This summary is not an extensive overview of the claimed subject matter. It is intended to neither identify key or critical elements of the claimed subject matter nor delineate the scope thereof. Its sole purpose is to present some concepts in a simplified form as a prelude to the more detailed description that is presented later.

The present disclosure provides an ankle joint mechanism, including a shank member, a first connecting link, a foot member, a second connecting link, and a linear force providing element. The shank member includes a distal portion and a proximal portion. The foot member is rotatably coupled to the distal portion of the shank member at a first pivot point and rotatably coupled to the first connecting link at a second pivot point. A distance between the first pivot point and the proximal portion of the shank member is less than a distance between the second pivot point and the proximal portion of the shank member. The second connecting link is rotatably coupled to the first connecting link at a third pivot point and rotatably coupled to the shank member at a fourth pivot point. The linear force providing element includes a first end and second end. The first end of the linear force providing element is coupled to the second connecting link, and the second end of the linear force providing element is coupled to a portion of the ankle joint mechanism other than the second connecting link.

In some examples, at least one of a distance from the proximal portion of the shank member to the third pivot point and a distance from the proximal portion of the shank member to the fourth pivot point is greater than a distance from the proximal portion of the shank member to the first pivot point.

In some examples, a distance from the proximal portion of the shank member to the second pivot point is greater than a distance from the proximal portion of the shank member to the fourth pivot point.

In some examples, the fourth pivot point is located between the first pivot point and the second pivot point.

In some examples, at least one of the first connecting link and the second connecting link are configured to be in tension when the ankle joint mechanism is under a dorsiflexive-inducing load.

In some examples, the linear force providing element is configured to be subject to a compressive force under a dorsiflexive-inducing load.

In some examples, the first end of the linear force providing element is rotatably coupled to the second connecting link at a fifth pivot point, and the second end of the linear force providing element is rotatably coupled to one of the shank member and the foot member at a sixth pivot point.

In some examples, the fourth pivot point is positioned on the second connecting link between the third pivot point and the fifth pivot point.

In some examples, one of the fourth pivot point, the first end of the linear actuator, or the second end of the linear force providing element includes a joint configured to rotate and to slide in a linear movement.

In some examples, the second end of the force providing element is coupled to one of the foot member and the shank member.

In some examples, the linear force providing element includes a first volume of working fluid, a second volume of working fluid, a piston, a valve, and at least one sensor. The valve connects the first volume of working fluid to the second volume of working fluid on opposing sides of the piston. The valve is configured to adjust a fluid flow between the first volume of working fluid and the second volume of working fluid. The at least one sensor is configured to vary a shape of the valve between at least two shapes. For example, a first shape of the valve increases the fluid flow and wherein a second shape of the valve decreases the fluid flow.

In some examples, the present disclosure provides an ankle prosthesis, including a shank member, a first connecting link, a foot member, a second connecting link, and a linear force providing element. The shank member includes a distal portion and a proximal portion. The foot member is rotatably coupled to the distal portion of the shank member at a first pivot point and rotatably coupled to the first connecting link at a second pivot point. A distance between the first pivot point and the proximal portion of the shank member is less than a distance between the second pivot point and the proximal portion of the shank member. The second connecting link is rotatably coupled to the first connecting link at a third pivot point and rotatably coupled to the shank member at a fourth pivot point. The linear force providing element includes a first end and second end. The first end of the linear force providing element is coupled to the second connecting link, and the second end of the linear force providing element is coupled to a portion of the ankle prosthesis other than the second connecting link.

In some examples, the ankle prosthesis further includes a foot cover. Each of the second pivot point, the third pivot point, and the fourth pivot point are positioned within the foot cover.

In some examples of the ankle prosthesis, at least one of the first connecting link, the second connecting link, and the linear force providing element is configured to measure torque applied to the prosthesis.

In some examples, the present disclosure provides an ankle orthosis, including a shank member, a first connecting link, a foot member, a second connecting link, and a linear force providing element. The shank member includes a distal portion and a proximal portion. The foot member is rotatably coupled to the distal portion of the shank member at a first pivot point and rotatably coupled to the first connecting link at a second pivot point. A distance between the first pivot point and the proximal portion of the shank member is less than a distance between the second pivot point and the proximal portion of the shank member. The second connecting link is rotatably coupled to the first connecting link at a third pivot point and rotatably coupled to the shank member at a fourth pivot point. The linear force providing element includes a first end and second end. The first end of the linear force providing element is coupled to the second connecting link, and the second end of the linear force providing element is coupled to a portion of the ankle orthosis other than the second connecting link.

Other features and characteristics of the subject matter of this disclosure, as well as the methods of operation, functions of related elements of structure and the combination of parts, and economies of manufacture, will become more apparent upon consideration of the following description and the appended claims with reference to the accompanying drawings, all of which form a part of this specification, wherein like reference numerals designate corresponding parts in the various figures.

### BRIEF DESCRIPTION OF THE DRAWINGS

The accompanying drawings, which are incorporated herein and form part of the specification, illustrate various embodiments of the subject matter of this disclosure. In the drawings, like reference numbers indicate identical or functionally similar elements.

FIG. 1 is a side view of an exemplary ankle prosthesis, according to the prior art.

FIG. 2 is a side view of an exemplary ankle joint mechanism, according to an embodiment of the present disclosure.

FIG. 3A is a side view of the exemplary ankle joint mechanism of FIG. 2 in a dorsiflexed position, according to an embodiment of the present disclosure.

FIG. 3B is a side view of the exemplary ankle joint mechanism of FIG. 2 in a plantarflexed position, according to an embodiment of the present disclosure.

FIG. 4A is a perspective view of the posterior, top, and left side of the ankle joint mechanism, according to an embodiment of the present disclosure.

FIG. 4B is a perspective view of the anterior, top, and left side of the ankle joint mechanism, according to an embodiment of the present disclosure.

FIG. 5 shows a schematic view of an exemplary linkage mechanism, according to an embodiment of the present disclosure.

FIG. 6 shows a schematic view of an exemplary linkage mechanism, according to an embodiment of the present disclosure.

FIG. 7 shows a schematic view of an exemplary linkage mechanism, according to an embodiment of the present disclosure.

FIG. 8 shows a schematic view of an exemplary linkage mechanism, according to an embodiment of the present disclosure.

FIG. 9 shows a schematic view of an exemplary linkage mechanism, according to an embodiment of the present disclosure.

FIG. 10 shows a perspective view of an exemplary second connecting link, according to an embodiment of the present disclosure.

FIG. 11 shows a perspective view of an exemplary first connecting link, according to an embodiment of the present disclosure.

FIG. 12 shows a perspective view of an exemplary view first connecting link coupled to a second connecting link, according to an embodiment of the present disclosure.

FIG. 13 shows a schematic view of an exemplary force providing element, according to an embodiment of the present disclosure.

### DETAILED DESCRIPTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

Unless defined otherwise, all terms of art, notations and other technical terms or terminology used herein have the same meaning as is commonly understood by one of ordinary skill in the art to which this disclosure belongs. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

Unless otherwise indicated or the context suggests otherwise, as used herein, “a” or “an” means “at least one” or “one or more.”

This description may use various terms describing relative spatial arrangements and/or orientations or directions in describing the position and/or orientation of a component, apparatus, location, feature, or a portion thereof or direction of movement, force, or other dynamic action. Unless specifically stated, or otherwise dictated by the context of the description, such terms, including, without limitation, top, bottom, above, below, under, on top of, upper, lower, left of, right of, in front of, behind, next to, adjacent, between, horizontal, vertical, diagonal, longitudinal, transverse, radial, axial, clockwise, counter-clockwise, etc., are used for convenience in referring to such component, apparatus, location, feature, or a portion thereof or movement, force, or other dynamic action in the drawings and are not intended to be limiting.

Furthermore, unless otherwise stated, any specific dimensions mentioned in this description are merely representative of an exemplary implementation of a device embodying aspects of the disclosure and are not intended to be limiting.

To the extent used herein, the term “adjacent” refers to being near or adjoining. Adjacent objects can be spaced apart from one another or can be in actual or direct contact with one another. In some instances, adjacent objects can be coupled to one another or can be formed integrally with one another.

To the extent used herein, the terms “substantially” and “substantial” refer to a considerable degree or extent. When used in conjunction with, for example, an event, circumstance, characteristic, or property, the terms can refer to instances in which the event, circumstance, characteristic, or property occurs precisely as well as instances in which the event, circumstance, characteristic, or property occurs to a close approximation, such as accounting for typical tolerance levels or variability of the embodiments described herein.

To the extent used herein, the terms “optional” and “optionally” mean that the subsequently described, component, structure, element, event, circumstance, characteristic, property, etc. may or may not be included or occur and that the description includes instances where the component, structure, element, event, circumstance, characteristic, property, etc. is included or occurs and instances in which it is not or does not.

The present disclosure provides an ankle joint mechanism, including a shank member, a first connecting link, a foot member, a second connecting link, and a force providing element. The foot member is coupled to the shank member at a first pivot point and coupled to the first connecting link at a second pivot point. The second connecting link is coupled to the first connecting link at a third pivot point and coupled to the shank member at a fourth pivot point. The force providing element is coupled to the second connecting link at a first end and coupled to either the shank member or the foot member at a second end. The disclosed ankle joint mechanism provides advantages compared to conventional ankle prostheses and orthoses by providing a design with less weight, a compact design configured to fit within an anthropomorphic design envelope of a prosthesis and/or orthosis, increased durability, and greater safety for users by concealing moving components within a cover, all without the corresponding deficits in performance of the ankle joint mechanism. The present disclosure contemplates the ankle joint mechanism discussed herein can be used in a prosthesis (that is, an artificial limb configured to replace a missing limb of the user) and/or an orthosis (that is, a device configured to correct, accommodate, or enhance the use of a limb of the user).

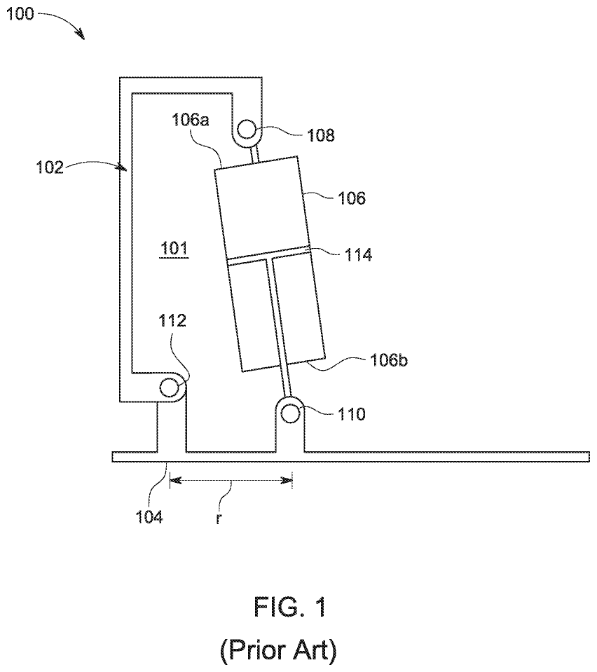

FIG. 1 is a side view of an exemplary slider-crank ankle joint mechanism 100, according to the prior art. Mechanism 100 includes a shank member 102, a foot member 104, a force providing element 106, a first connection point 108, a second connection point 110, and a rotary joint 112.

Shank member 102 is coupled to foot member 104 both at rotary joint 112 and via force providing element 106. For example, the rotary joint 112 allows the foot member 104 to rotate relative to the shank member 102. The force providing element 106 is coupled to the shank member 102 at first connection point 108 and is coupled to the foot member 104 at second connection point 110.

The force providing element 106 is typically either an electromechanical actuator or a hydraulic actuator. In both cases, the force providing element may be incorporated into a slider-crank mechanism 100, which transduces actuation force to ankle torques.

A commonly-used transmission mechanism for the design of an articulated ankle prosthesis is that of a slider-crank mechanism 100 as shown in FIG. 1. For example, as shown in FIG. 1, the force providing element 106 is coupled at one end 106a via a rotary joint (e.g., connection point 108) to the shank member 102 while the other end 106b of the force providing element 106 is coupled via a rotary joint (e.g., connection point 110) to the foot member 104. The slider-crank mechanism 100 transduces the linear force and motion of the force providing element 106 into the torque that rotates foot member 104 about rotary joint 112. In such a design, the ratio of ankle torque (defined as τ) to actuator (linear) force (defined as F) is directly proportional to the lever arm of the slider-crank mechanism (defined as r, and shown in FIG. 1), as expressed by Equation 1 below:

$$\frac{τ}{F} ∝ r \qquad Equation 1$$

In such a configuration, for the same ankle torque, the required actuator force becomes smaller as the lever arm r becomes larger. Small actuation forces allow for the use of a small and lightweight force providing element 106. However, as the lever arm r of the slider-crank mechanism becomes larger, so too does the design envelope of the mechanism 100 (that is, r is proportional to the size of the mechanism 100 because an increase in r increases the interior space 101 of the mechanism 100). A larger design envelope typically equates to a bulky prosthesis that does not match the anthropomorphic characteristics of a human ankle. Accordingly, the designs of conventional prostheses must trade off between the size of the lever arm and the size of the force providing element 106. As a result of this tradeoff, articulated prosthetic ankles utilizing the slider-crank mechanism 100 are frequently subject to very high forces by the force providing element 106 (in order to maintain an anthropomorphic design envelope), necessitating the need for large force providing elements 106. A large force providing element 106 presents its own challenges to maintain an anthropomorphic design envelope because a large force providing element 106 increases the weight of the prosthesis and required volume of the shank portion.

In some examples, conventional prosthetic ankle devices using the transmission mechanism 100 opt to limit the ankle torque that the mechanism 100 can sustain by providing smaller force providing elements 106 and smaller lever arms r in order to maintain a small form factor; however, this limits the biomechanical functionality of the device.

Therefore, mechanism 100 does not provide a transmission mechanism that allows for a prosthetic ankle to sustain large ankle torques with low actuation force while simultaneously maintaining a compact, anthropomorphic design envelope.

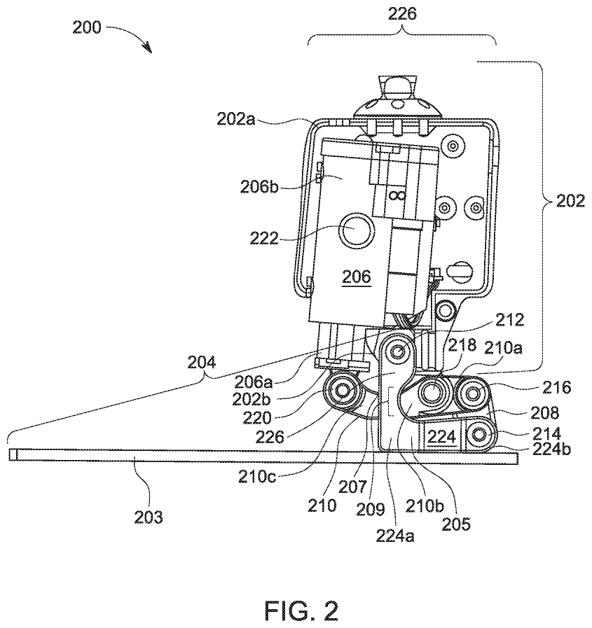

To overcome the shortcomings of conventional ankle prostheses, the present disclosure provides an ankle joint mechanism. FIG. 2 is a side view of an exemplary ankle joint mechanism 200 in a resting position on flat terrain, according to an embodiment of the present disclosure. Ankle joint mechanism 200 includes a shank member 202, a foot member 204 optionally including a base portion 203 and an anchor portion 205, a force providing element 206, a first connecting link 208, a second connecting link 210, a first pivot point 212, a second pivot point 214, a third pivot point 216, a fourth pivot point 218, a connection point 220, and a sixth pivot point 222.

A shank member 202 is coupled to foot member 204. In some examples, the shank member 202 is configured to receive a lower limb portion of a user and corresponds anthropomorphically to a human leg shank. The shank member includes a proximal portion 202a located towards the top of the of the ankle joint mechanism 200 and a distal portion 202b located towards bottom of the ankle joint mechanism 200, adjacent to the foot member 204. For example, the shank member is configured to have a cavity in its interior that can contain parts of the ankle joint mechanism. For example, the shank member contains the sixth pivot point 222, thereby concealing this component within the body of the shank member. Concealing rotating pivot points such as 222 helps to increase user safety by preventing clothing from becoming caught between moving components.

In some examples, the foot member 204 receives weight from the user during movement, and, in some examples, the foot member 204 corresponds anthropomorphically to a human foot. For example, the foot member 204 may include a base portion 203, which interacts with the terrain as a user moves, and an anchor portion 205, which extends above the base portion 203 towards the shank member 202 and is configured to couple with the shank member 202. For example, the anchor portion 205 may comprise an L-shaped link with a first leg 224 fixed to the base portion 203 proximate one end thereof and a second leg 226 extending from a first end portion 224a of the first leg 224. For example, the first end portion 224a of the first leg 224 is opposite from a second end portion 224b. In some examples, the length of the first leg 224 of the anchor portion 205 is approximately half the width 226 of the shank member 202.

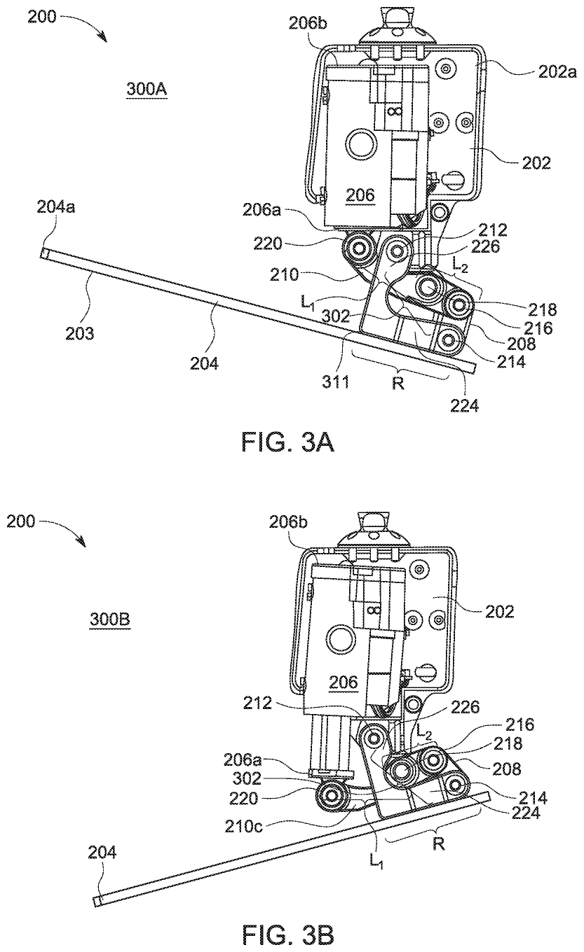

The foot member 204 is rotatably coupled to the distal portion 202b of the shank member 202 at first pivot point 212 on the second leg 226 of the anchor portion 205. The first pivot point 212 corresponds to the anthropomorphic ankle joint and provides an axis of rotation about which the ankle joint mechanism 200 dorsiflexes and plantarflexes under corresponding loads (see, e.g., FIGS. 3A and 3B). The foot member 204 is further rotatably coupled to first connecting link 208 at second pivot point 214 on the first leg 224 of the anchor portion 205. A distance between the first pivot point 212 and the proximal portion 202a of the shank member 202 is less than a distance between the second pivot point 214 and the proximal portion 202a of the shank member 202.

In some examples, the pivot points 212, 214 are located anywhere along the first leg 224 and second leg 226, respectively. For example, pivot points 212, 214 can be located closer to first end portion 224a. As discussed further regarding FIG. 3A below, a lever arm R of the ankle joint mechanism 200 is the distance between first pivot point 212 and second pivot point 214 along the first leg 224. As 214 moves inward along the first leg 224, towards the first end portion 224a, the lever arm R decreases, and a transmission ratio gets smaller (as shown and discussed further below regarding Equation 2).

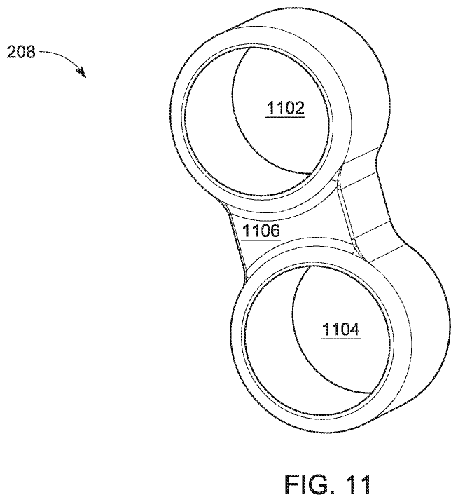

Turning briefly to FIG. 11, first connecting link 208 includes a first receiving portion 1102, a second receiving portion 1104, and a body portion 1106. For example, as discussed further below, first connecting link 208 is a two-force member and substantially symmetrical along a longitudinal axis.

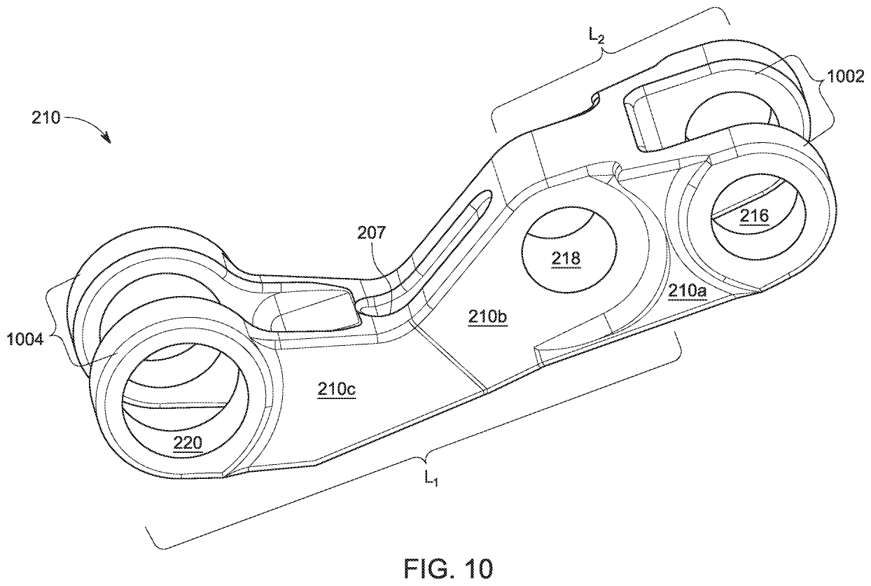

Turning now to FIG. 10, an angled perspective view of second connecting link 210 is shown. In some examples, the second connecting link 210 has a bowed, asymmetric, and/or angular shape. For example, the second connecting link 210 includes first portion 210a extending from the third pivot point 216 to the fourth pivot point 218, second portion 210b extending from the fourth pivot point 218 to an inflection point 207, and third portion 210c extending from inflection point 207 to connection point 220. In some examples, the second portion 210b is wider at the fourth pivot point 218 than at the inflection point 207. For example, the shape of the second connecting link 210 is provided such that the second connecting link 210 moves through its range of motion without the first portion 210a, second portion 210b, and third portion 210c contacting the other components of the ankle joint mechanism. The second connecting link 210 further includes receiving portions 1002 and 1002.

In alternate examples (not shown), the second connecting link 210 has a V-shape or an L-shape with inflection points at point 207. In alternate examples (not shown), pivot points 216, 218, and 220 are collinear and the second connecting link 210 is a straight link.

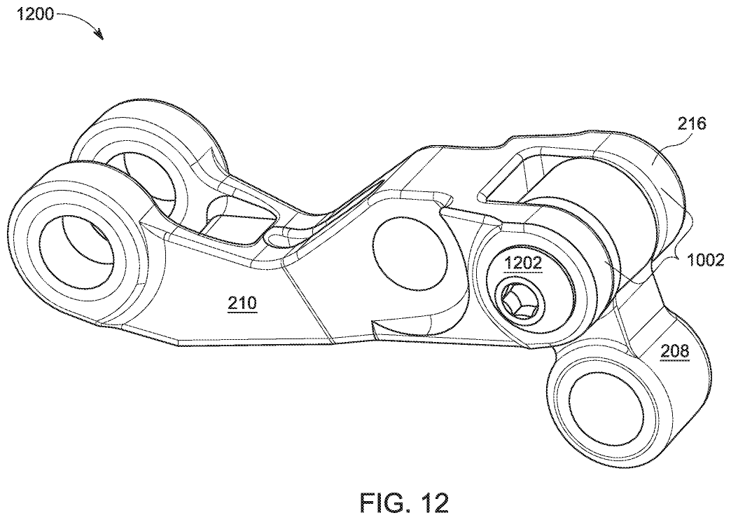

As shown in FIG. 12, receiving portion 1002 is configured to rotatably couple to the first connecting link 208 at third pivot point 216 (e.g., through one of receiving portions 1102, 1104 of first connecting link 208). In some examples, the first connecting link 208 is coupled to the second connecting link 210 by a bolt or other mechanical coupler. For example, as shown by FIGS. 2-4B, connecting portion 1004 is configured to receive the first end 206a of the force providing element 206.

Turning back to FIG. 2, the second connecting link 210 is rotatably coupled to the shank member 202 at fourth pivot point 218. The second connecting link 210 is further coupled to the force providing element 206, for example, at connection point 220. For example, the second connecting link 210 is oriented at an angle relative to the force providing element 206 in the range of 45 degrees to 135 degrees and preferably in the range of 60 degrees to 120 degrees.

Turning back to FIG. 2, the first portion 210a extends substantially parallel to the first leg 224 of the anchor portion 205. The second portion 210b may be angled downwards towards the base portion 203 of the foot member 204 such that inflection point 207 is closer to the base portion 203 of the foot member than the fourth pivot point 218. In some examples, the third portion 210c is angled upwards towards the proximal end portion 202a of the shank member 202 such that the connecting point 220 is closer to the shank member 202 than the inflection point 207. For example, the third portion 210c and the second portion 210b are oriented relative to each other at an obtuse angle in the range of 170 degrees to 150 degrees.

In some examples, the second connecting link 210 is oriented transversely to the first connecting link 208 when the ankle joint mechanism 200 is in a resting position shown in FIG. 2 (i.e., neither under a plantarflexive-inducing load nor under a dorsiflexive-inducing load). For example, the second connecting link 210 is oriented at an angle relative to the first connecting link 208 in the range of 45 degrees to 135 degrees and preferably in the range of 60 degrees to 120 degrees. In examples where the second connecting link 210 has a bowed, asymmetric, and/or angular shape, the angle relative to the first connecting link 208 is determined based on an axis extending between the third pivot point 216 and the fourth pivot point 218.

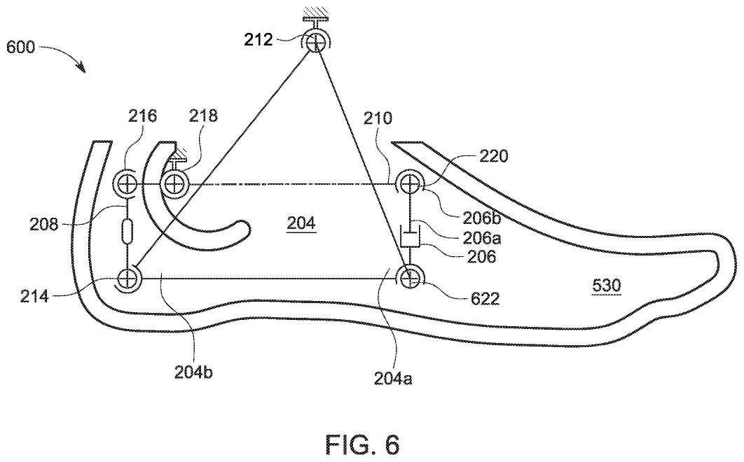

Ankle joint mechanism 200 further includes force providing element 206 with a first end portion 206a and a second end portion 206b. For example, the second end portion 206b includes a cylinder or barrel portion of the force providing element 206 and the first end portion 206a includes a rod which extends from and compresses into the second end portion 206b. The first end portion 206a is coupled to the second connecting link 210. In some examples, the second end portion 206b is coupled to the shank member 202. For example, the coupling between the second end portion 206b and the shank member 202 can be static, or the coupling between the second end portion 206b and the shank member 202 can be a sixth pivot point 222. In alternate examples, as shown and discussed further regarding FIG. 6, the second end portion 206b is coupled to the foot member 204 instead of to the shank member 202.

In some examples, the first end portion 206a is rotatably coupled to the second connecting link 210 at connection point 220, which comprises a fifth pivot point; the fourth pivot point 218 is thus positioned on the second connecting link 210 between the third pivot point 216 and connection point 220. In some examples, the fourth pivot point 218 is positioned closer to the third pivot point 216 than to the connection point 220.

In some examples, the force providing element 206 is at least one of: a linear force providing element, a linear actuator, a power screw assembly comprising an electric motor, a voice coil motor, a linear motor, a spring, a magnetorheological damper, a electrorheological damper, a pneumatic actuator, and a hydraulic actuator. Hydraulic actuators exhibit higher force/torque density than the force/torque density of electromechanical actuators, and, consequently, hydraulic actuators are useful for prostheses and orthoses. Hydraulic actuators may also be divided into two classes: rotary and linear. Of these two classes of hydraulic actuators, the linear variant exhibits larger force/torque density and fewer fluid leakage issues due to the high performance of linear hydraulic seals relative to rotary sealing technology. Therefore, the force providing element 206 is configured to generate a force between the first end portion 206a and the second end 206b of the force providing element 206.

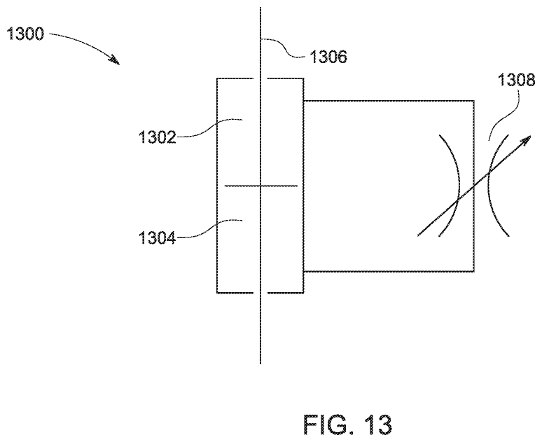

Turning briefly to FIG. 13, a schematic drawing of an exemplary force providing element 1300 is shown, according to an embodiment of the present disclosure. In some examples, the force providing element 1300 includes a first volume of working fluid 1302, a second volume of working fluid 1304, a piston 1306, and an adjustable flow control valve 1308. For example, the valve 1308 is configured to control the flow of fluid between the first volume of working fluid 1302 and the second volume of working fluid 1304 on opposing sides of the piston 1306. In some examples, the force providing element 206 further comprises at least one sensor configured to vary a shape of the valve 1308 between at least two shapes, wherein a first shape of the valve increases the fluid flow and wherein a second shape of the valve decreases the fluid flow. In some examples, the valve 1308 is electronically adjusted by a computing device communicatively coupled to the sensor; accordingly, said computing device controls the position of the valve 1308 and/or a pressure drop across the valve between the first volume of working fluid 1302 and the second volume of working fluid 1304. In some examples, the valve 1308 can be manually adjusted by a user of the ankle joint mechanism in which the force providing element 1300 is housed.

In some examples, a sensor measures a resistance level and/or position of the valve 1308. In some examples, the sensor is a potentiometer or an encoder, which measures the position of the valve 1308 to determine a measure of the valve orifice size (through which fluid flows). For example, measurement of valve orifice size is used to determine information regarding the resistance (i.e. damping) provided by the valve 1308. In other examples, the sensor is a pressure gauge, which measures pressure drop across the valve 1308. For example, measurement of pressure drop is used to determine information about the resistance being provided by the force providing element 1300 as a result of the valve 1308.

In some examples, the valve 1308 is adjusted to restrict flow between the two volumes of working fluid 1302, 1304 during the ground contact and stance phases of walking. During the ground contact phase of walking, the movement of the ankle mechanism (e.g., mechanism 200 of FIG. 2) should be highly damped, and accordingly, the valve 1308 can be positioned as to heavily restrict flow between the two volumes of working fluid 1302, 1304. During the stance phase of walking, the ankle mechanism (e.g., mechanism 200 of FIG. 2) should provide support to the user. Accordingly, the valve 1308 can be positioned as to heavily restrict or disallow flow between the two volumes of working fluid 1302, 1304. In such a valve configuration, the ankle mechanism (e.g., mechanism 200 of FIG. 2) provides torques that support the weight of the user. During the swing phase of gait, the ankle may be repositioned to provided ground clearance for the user (i.e. the toe may be lifted to avoid stumble hazards). To allow this repositioning to occur, the valve 1308 may be positioned so as to readily allow flow between the two volumes of working fluid 1302, 1304. Accordingly, the valve 1308 may be positioned so as to allow for movement of the ankle mechanism (e.g., mechanism 200 of FIG. 2) with little resistance. For example, the different phases of gait are determined by a number of sensors housed on the ankle joint mechanism (e.g., mechanism 200 of FIG. 2) such as load sensors, inertial measurement units (IMUs), joint encoders, and other sensors as would be readily identified by one skilled in the art.

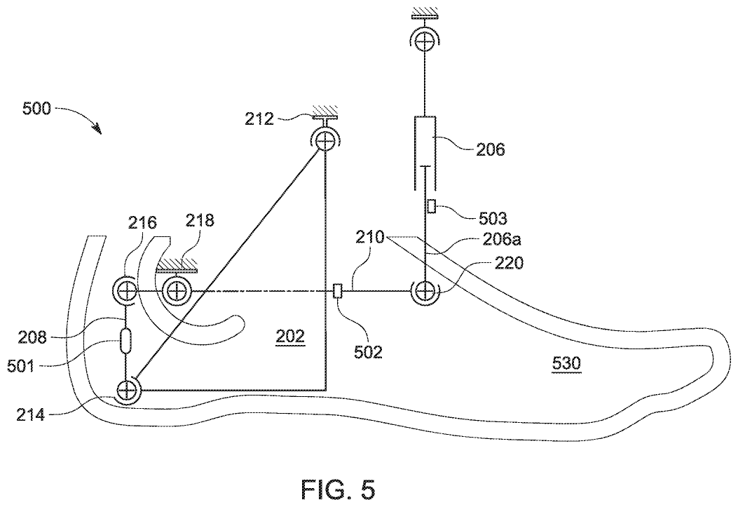

Furthermore, in some examples, a position of the valve 1308 is changed in the ankle joint mechanism (e.g., mechanism 200 of FIG. 2) depending the phase of gait being performed by the user. For example, load sensors are used to determine which phase of gait is being performed (see, e.g., FIG. 5 and the corresponding description below). For example, the valve 1308 adopts a high resistance configuration during stance phase and a low resistance configuration during swing phase. The transition between stance and swing phase may be measured by detecting when the user has unloaded the ankle joint mechanism (e.g., mechanism 200 of FIG. 2). This unloading may be measured by estimating the torque applied to the ankle through the use of a load sensor on the first connecting link, second connecting link, or force providing element (see, e.g., FIG. 5 and the corresponding description below).

Turning back to FIG. 2, the ankle joint mechanism 200 provides a four-bar linkage (that is, the four bars are (1) the foot member 204, (2) the first connecting link 208, (3) the second connecting link 210, and (4) the shank member 202) with at least four pivot points. The exemplary ankle joint mechanism 200 further provides a slider-crank mechanism consisting of the force providing element 206, the shank member 202, and the second connecting link 210. Therefore, the ankle joint mechanism 200 provides a two-stage linkage transmission (e.g., the first stage is the slider-crank mechanism and the second stage is the four-bar linkage), where movement of the four-bar linkage, and the ankle joint mechanism 200 as a whole, is driven by the slider-crank mechanism.

The majority of the four-bar linkage mechanism is located below the ankle joint (e.g., the first pivot point 212) and above the foot member 204. Specifically, the second pivot point 214, third pivot point 216, fourth pivot point 218 are located below the first pivot point 212, and therefore inferior (i.e., lower in position) to the ankle joint. Additionally, if applicable, the fifth pivot point 220, is also located below the first pivot point 212. For example, a distance from the proximal portion 202a of the shank member 202 to the third pivot point 216, or a distance from the proximal portion 202a of the shank member 202 to the fourth pivot point 218, is greater than a distance from the proximal portion 202a of the shank member 202 to the first pivot point 212. For example, a distance from the proximal portion 202a of the shank member 202 to the second pivot point 214 is greater than a distance from the proximal portion 202a of the shank member 202 to the fourth pivot point 218.

This arrangement of the foot member 204, the first connecting link 208, the second connecting link 210, and the shank member 202 allows for the four-bar linkage mechanism to have a low build height (further minimizing the size of the ankle joint mechanism 200) as the four-bar linkage mechanism can be packaged between the ankle joint (e.g., first pivot point 212) and the base portion 203 of the foot member 204. Furthermore, the arrangement of the first pivot point 212, second pivot point 214, and fourth pivot point 218 in the ankle joint mechanism 200 causes the second leg 226 of the anchor portion 205 of the foot member 204 to overlap with the second connecting link 210 (e.g., shown in region 207) during all movement of the ankle joint mechanism 200. For example, the overlap at region 207 occurs at a portion of the second connecting link 210 between the second portion 210b and the third portion 210c of the second connection link 210. This overlap of the first link (i.e., the foot member 204) and the third link (i.e., the second connecting link 210) in the four-bar linkage mechanism in region 207 further helps to conserve space and improve the compactness of the ankle joint mechanism 200.

The ankle joint mechanism 200 ensures greater user safety than conventional prostheses. Safety is a driving factor in the design of prosthetic devices because clothes or body parts (e.g., fingers) can be caught in the “pinch points” of the multiple moving components. In an ankle prosthesis, into which the ankle joint mechanism 200 is integrated in some embodiments of the present disclosure, a cosmetic foot cover is used in conjunction with the foot member 204 (for example, shown schematically in FIGS. 5-8) and a shell surrounds the shank member 202. The disclosed ankle joint mechanism 200 includes all moving components beneath the first pivot point 212, near the foot member 204, such that a cosmetic foot cover conceals the four-bar linkage components. That is, in some examples, each of the second pivot point 214, third pivot point 216, fourth pivot point 218, and, if applicable, the fifth pivot point 220, are positioned within a foot cover. This positioning eliminates the user safety risk posed by pinch points. In fact, the design of the four-bar linkage mechanism is so compact that, in some examples, it is placed within the anthropomorphic envelope of a healthy ankle. Moreover, by concealing the four-bar linkage mechanism within a cosmetic foot cover, space within the shell of the shank member 202 may then be used for other components such as batteries.

Besides the benefits which have already been discussed herein, the slider-crank mechanism and the four-bar linkage of the two-stage linkage transmission provide several advantages that conventional ankle prostheses are unable to achieve. For example, the two-stage linkage transmission of ankle joint mechanism 200 further provides for a small, compact design envelope of the ankle joint mechanism 200, and the design envelope is kept compact by means other than just the increased mechanical advantage of the ankle joint mechanism. Also, the four-bar linkage provides for the force providing element 206 to be positioned generally in-line with the shank member 202, allowing a larger actuator to remain within the anthropomorphic envelope. Additionally, the disclosed two-stage transmission mechanism provides for a single axis of rotation between the shank member 202 and foot member 204, in contrast to most conventional four-bar linkages that typically produce a moving center of rotation for the ankle joint (remote/instantaneous center mechanisms).

Such a two-stage transmission mechanism provides for the use of a small, lightweight force providing element 206 while still maintaining the ability to provide the appropriate torque about the ankle joint. Small device size promotes acceptance of the prosthesis by the user, with the upper limits of the size of the ankle joint mechanism 200 roughly determined by the anthropomorphic envelope defined by the anatomical ankle and foot. Such design requirements are necessary with regard to fitting within existing shoes and other clothing as well as fitting patients of small stature or patients with long residual limbs.

The construction of the ankle joint mechanism 200 is further arranged to maximize durability. Durability in an ankle prosthesis is largely dependent on the failure modes of the various components. In prosthetic ankles, loading is very large in one direction (dorsiflexion) and relatively small in the other direction (plantarflexion). Therefore, as discussed regarding FIGS. 3A and 3B below, the components of the ankle joint mechanism 200, and particularly the first connecting link 208 and force providing element 206 which receive said high forces during dorsiflexion, are arranged to withstand high forces when loaded in the direction corresponding to high ankle loads.

FIG. 3A is a side view of the exemplary ankle joint mechanism 200 of FIG. 2 in a dorsiflexed position 300A, according to an embodiment of the present disclosure. FIG. 3B is a side view of the exemplary ankle prosthesis of FIG. 2 in a plantarflexed position 300B, according to an embodiment of the present disclosure. Similar reference labels in FIGS. 3A-3B correspond to elements in FIG. 2. FIGS. 3A-3B demonstrate movement and relevant characteristics of the ankle joint mechanism 200.

The second connecting link 210 is configured to see-saw generally about the fourth pivot point 218, where the second connecting link 210 is rotatably coupled to the shank member 202, while the ankle joint mechanism 200 transitions between a dorsiflexed position 300A and a plantarflexed position 300B. Therefore, the fourth pivot point 218 moves in a region of space 302 between the first pivot point 212 and the second pivot point 214.

When the ankle joint mechanism 200 is under a dorsiflexion-inducing load, as shown in FIG. 3A, the end portion 204a of the foot member 204 is rotated upwards about an axis through the first pivot point 212, towards the proximal portion 202a of the shank member 202. The first end portion 206a of the force providing element 206 (partially extended in FIG. 2) is compressed within the body of the force providing element 206. In some examples, the force providing element 206 is a hydraulic actuator. Hydraulic actuators, however, are subject to fluid leakage when subject to high pressures and commonly leak at the sealing around the rod of the hydraulic actuator. Hydraulic actuator rod seals are most vulnerable to leakage when the hydraulic actuator is under tension, thereby placing the rod side of the cylinder under high pressure. As discussed above, loading on the human ankle is asymmetric, with large dorsiflexive loading and low plantarflexive loading. As such, to reduce the likelihood of hydraulic leakage around the rod seals, the force providing element 206 is under compression when subject to high loads, thereby minimizing the likelihood of fluid leakage when the force providing element 206 is a hydraulic actuator.

Further, the second connecting link 210 is configured to see-saw about the fourth pivot point 218 when moving between dorsiflexed position 300A and plantarflexed position 300B. When the ankle joint mechanism 200 transitions into the dorsiflexed position 300A from a resting position or from plantarflexed position 300B (or, simply, when the base portion 203 of the foot member 204 receives a force/torque pushing the end portion 204a of the foot member 204 upwards to the shank member 202), the second connecting link 210 is configured to pivot clockwise about the fourth pivot point 218. The third pivot point 216 is accordingly pulled down by the first connecting link 208, which is attached to the first leg 224 of the anchor portion 205 by the second pivot point 214. The second pivot point 214 and accordingly the first connecting link 208 is pulled downward by the force/torque acting on the base portion 203, putting the first connecting link 208 in tension. In some examples, the first connecting link 208 is a two-force member, and, accordingly, all forces through the first connecting link 208 act in a straight line between the second pivot point 214 and the third pivot point 216. Two-force members loaded in tension only fail if the stresses acting on the member exceed the material strength. However, when two-force members are loaded in compression, they may fail either by the stress on the part exceeding the material strength or by buckling under load. In order to eliminate the buckling failure mode, the two-force member should not be substantially loaded in compression. Because the first connecting link 208 is in tension when the ankle joint mechanism 200 is subjected to high dorsiflexive loading, the ankle joint mechanism 200 avoids the buckling failure possibility for the first connecting link 208.

When the ankle joint mechanism 200 includes a fifth pivot point at connection point 220 and is in position 300A, a distance from the fifth pivot point to an intersection 311 of the anchor portion 205 and the base portion 203 is substantially similar to a distance from the first pivot point 212 to intersection 311 (that is, the distances are within 0.5 inches of each other, and preferably, within 0.1 inches of each other).

When the ankle joint mechanism 200 is under a plantarflexive-inducing load, as shown in FIG. 3B (or, simply, when the base portion 203 of the foot member 204 receives a force/torque pushing the end portion 204a of the foot member 204 downwards away from the shank member 202), the first end portion 206a of the force providing element 206 extends from the second end portion 206b, and the second connecting link 210 pivots in a counter-clockwise movement about the fourth pivot point 218. Therefore, the force providing element 206 is under tension while the mechanism 200 experiences a plantarflexive load 300B. Additionally, the second connecting link 210 moves downward towards the base portion 203 of the foot member 204 such that the first connecting link 208 is compressed and positioned at an acute angle relative to the base portion 203 of the boot member 204.

In the ankle joint mechanism 200, the ratio of ankle torque to actuator force is directly proportional to the lever arm R between the ankle joint (e.g., the first pivot point 212) and the second pivot point 214 multiplied by the ratio of a distance L₁ between fourth pivot point 218 and the first end 206b of the force providing element 206 to the distance L₂ between the third pivot point 216 and the fourth pivot point 218 (see Equation 2 below). In the ankle joint mechanism 200, R is proportional to the size of the ankle joint mechanism 200, but the ratio of L₁ to L₂ can be made larger without a corresponding increase in the size of the ankle joint mechanism 200. In a preferred embodiment, L₁ is larger than L₂, making the ratio of L₁ to L₂ greater than one. The see-saw-like mechanism created by the second connecting link 210 and its corresponding pivot points 216, 218 allows for the ratio of ankle torque to actuator force to be made large while simultaneously maintaining a small design envelope of the ankle joint mechanism 200. Comparing the ankle joint mechanism 200 to a conventional slider-crank mechanism (e.g., the slider-crank mechanism shown by mechanism 100 of FIG. 1), the lever arms r and R both affect the size of the corresponding prostheses in comparable ways. However, with ankle joint mechanism 200, the ankle torque to actuator force ratio

$$( \frac{τ}{F} )$$

can be made larger by a factor of

$$\frac{{L}_{1}}{{L}_{2}} .$$

This multiplicative factor allows for actuator forces much lower than the typical slider-crank mechanism (e.g., the slider-crank mechanism shown by mechanism 100 of FIG. 1) without any corresponding increase in size of the ankle joint mechanism 200.

$$\frac{τ}{F} ∝ R \frac{{L}_{1}}{{L}_{2}} \qquad Equation 2$$

FIGS. 3A-3B further show that the angle of the force providing element 206 relative to the shank member 202 changes very little throughout the range of motion of the ankle joint mechanism 200, allowing other components housed within the shank member 202 to be packaged closely to the force providing element 206, further minimizing the design envelope.

Additionally, the angular range of motion of the force providing element 206 is smaller than conventional prostheses. The traditional slider-crank mechanism (as shown in FIG. 1) sweeps over a significant volume throughout its range of motion. The swept angle of the force providing element (e.g., elements 106 of FIG. 1 or 206 of FIG. 2) in a slider-crank mechanism is the range of angles between the force providing element and the shank member (e.g., elements 102 of FIG. 1 or 202 of FIG. 2) across the mechanism's full range of motion. This swept angle is directly proportional to the length of the crank arm of the slider-crank mechanism for a given output range of motion. In the proposed mechanism, the crank arm of the slider-crank portion of the mechanism is L₁, while in a standard ankle designed with a slider-crank mechanism, the crank length is r. Therefore, if the length of L₁ in the proposed mechanism is less than r in a standard slider-crank configuration for the same output range of motion and torque-to-force ratio (τ/F made equal), then the swept angle of the force providing element will be smaller with the proposed mechanism of FIGS. 2-3B than the swept angle of conventional prostheses, as shown in FIG. 1. Minimizing this swept angle accordingly minimizes the size of the ankle joint mechanism 200.

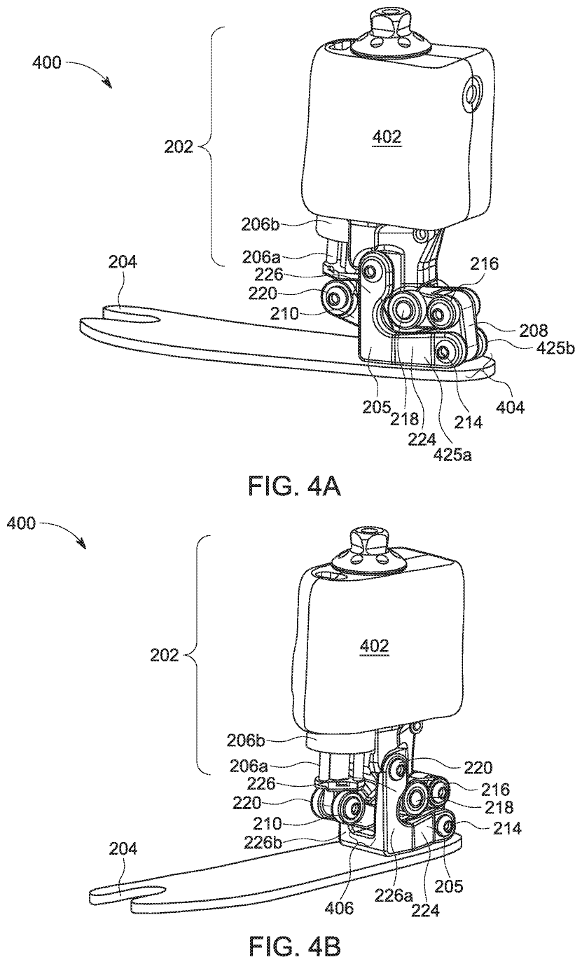

FIG. 4A is a perspective view of the anterior, top, and left side of an ankle joint mechanism 400, according to an embodiment of the present disclosure. FIG. 4B is a perspective view of the posterior, top, and left side of the ankle joint mechanism 400, according to an embodiment of the present disclosure. Similar reference labels in FIGS. 4A-4B correspond to elements in FIG. 2-3B. FIGS. 4A-4B further include a shank shell portion 402.

As shown in these perspective views, in some examples, the first leg 224 and the second leg 226 of the anchor portion 205 of the foot member 204 are u-shaped, including cutout region 404 that defines spaced-apart, left and right first leg segments 425a and 425b, as shown in in FIG. 4A and cutout region 406 that defines spaced-apart, left and right second leg segments 226a and 226b in FIG. 4B. Thus, in this example, the first connecting link 208 couples to the first leg 224 at the second pivot point 214 between left first leg segments 425a and right first leg segment 425b of the first leg 224. Second connecting link 210 extends through cutout region 406 between left and right second leg segments 226a and 226b, and shank member 202 couples to the connection point 220 between left and right second leg segments 226a and 226b. Accordingly, forces transferred between shank member 202 and foot member 204 and between first connecting link 208 and second connecting link 210 and foot member 204 are evenly distributed laterally across the foot member 204 so as to avoid lateral twisting of the foot member 204 with respect to the shank member 202 and the first connecting link 208 and second connecting link 210.

The shell 402 is configured to conceal components housed on or within the shank member 202, including the force providing element 206, a battery, and other electronic components. The shell 402 encloses components in order to minimize the risk posed by exposed pinch points associated with moving components. Furthermore, the shell 402 conceals electronic components, batteries, and wires, thereby protecting these components from damage.

FIGS. 5-8 show schematics of alternative configurations of the four-bar linkage mechanism, according to various embodiments of the present disclosure. In some examples, the second connecting link 210 of the ankle joint mechanisms of FIGS. 5-8 is positioned behind the foot member 502 (e.g., into the page) or passing through the foot member 502; this is shown by dashed lines.

FIG. 5 shows a schematic view of an exemplary linkage mechanism 500, according to an embodiment of the present disclosure. Similar reference labels in FIG. 5 correspond to elements in FIG. 2-4B. Ankle joint mechanism 500 further includes first sensor 501, second sensor 502, third sensor 503, and foot cover 530.

The first connection link 208 includes first sensor 501. For example, the first sensor 501 is positioned anywhere along a body of the first connection link 208 between the second pivot point 214 and the third pivot point 216. The second connection link 210 includes second sensor 502. For example, the second sensor 502 is positioned anywhere along a body of the second connection link 210 between the third pivot point 216 and the connection point 220. The force providing element 206 includes third sensor 503. For example, the third sensor 503 is positioned anywhere along the first end 206a of the force providing element 206. In some examples, any of the first sensor 501, the second sensor 502, and the third sensor 503 are strain gauges calibrated to measure force, pressure, tension, and/or weight. The first sensor 501 measures force, pressure, tension, and/or weight applied to the first connection link 208 during movement. The second sensor 502 measures force, pressure, tension, and/or weight applied to the second connection link 210 during movement. The third sensor 503 measures force, pressure, tension, and/or weight applied to the force providing element 206 during movement. In some examples, the loading data collected by any of the first 501, second 502, or third sensor 503 is used to determine the torque applied to the ankle. As such, this information may be used to transition between states and/or positions during walking as the prosthesis user loads and unloads the device. The data collected by this sensor can also be used to monitor how a prosthesis user utilizes their device (i.e., how many steps they take or which activities they are performing).

Foot cover 530 corresponds to an anthropological foot shape and is configured to cover various elements of ankle joint mechanism 500, including the first connecting link 208, the second connecting link 210, and at least part of the foot member 202.

FIG. 6 shows a schematic view of an exemplary ankle joint mechanism 600 where the second end 206b of the force providing element 206 is coupled to the second connecting link 210 and the first end 206a of the force providing element 206 is coupled to the foot member, according to an embodiment of the present disclosure. Similar reference labels in FIG. 6 correspond to elements in FIG. 2-5. Ankle joint mechanism 600 further includes a pivot point 622.

For example, the first end portion 206a of the force providing element 206 is rotatably coupled to the foot member 204 at pivot point 622, which is located towards a first end 204a of the foot member 204. First end 204a corresponds to a portion of the foot member 204 closer to the anthropological toes and second end 204b corresponds to a portion of the foot member 204 closer to the anthropological heel. For example, pivot point 622 forms a sixth pivot point of the ankle joint mechanism 600. Accordingly, the force providing element 206 is configured to fit within foot cover 530. When the ankle joint mechanism 600 is subject to a dorsiflexive-inducing load, the first connecting link 208 is placed in tension, as was previously discussed regarding ankle joint mechanism 200. However, in contrast to the loading of the force providing element 206 in ankle joint mechanism 600, the force providing element 206 is placed under tension instead of compression when the ankle joint mechanism 600 is placed under a dorsiflexive-inducing load. In the case where the force providing element 206 is not a fluid-driven actuator (i.e., the force providing element 206 is a power screw actuator), such a load distribution (both the first connecting link 208 and the force providing element 206 under tension during dorsiflexion-inducing loads) is advantageous to minimize component failure modes. Ankle joint mechanism 600 further provides a smaller design envelope, and further reduces pinch points of the ankle joint mechanism 600.

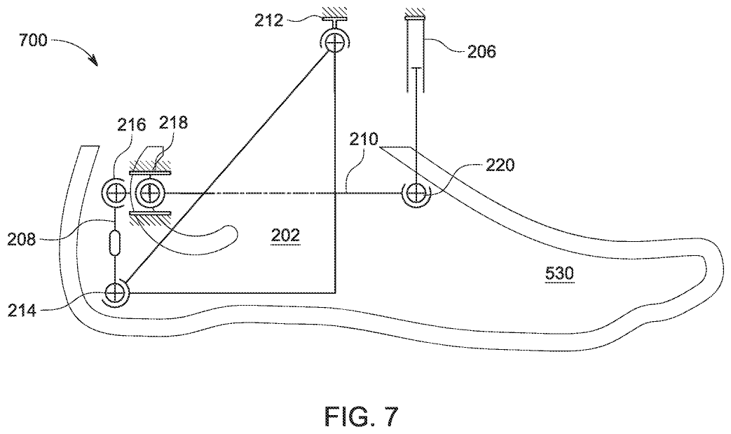

FIG. 7 shows a schematic view of an exemplary ankle joint mechanism 700, according to an embodiment of the present disclosure. Similar reference labels in FIG. 7 correspond to elements in FIG. 2-5. In the ankle joint mechanism 700, the second connecting link 210 is able to rotate about the fourth pivot point 218. Additionally, the fourth pivot point 218 is able to slide relative to the shank member 202 in a direction substantially parallel to the second connecting link. As the force providing element 206 extends and retracts, the fourth pivot point 218 slides anteriorly and posteriorly (e.g., towards and away from the anthropological toes) along a surface that defines the sliding constraint (pictured in FIG. 7 as parallel grounded walls).

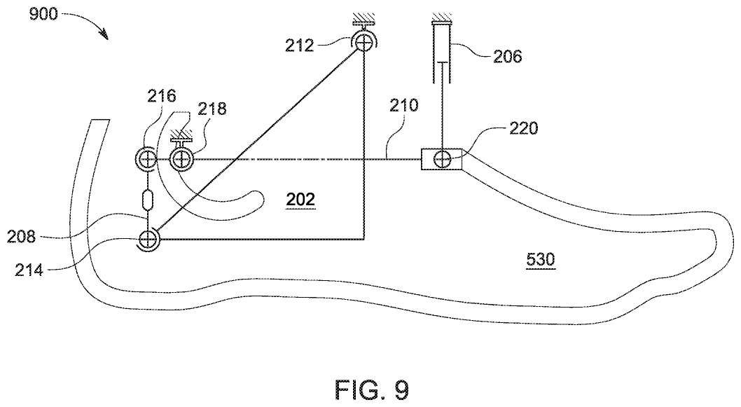

Therefore, the present disclosure contemplates that, in some embodiments, one of the fourth pivot point 218, the first end portion 206a of the force providing element 206, or the second end portion 206b of the force providing element 206 is a joint configured to rotate and to slide in a linear movement. Another exemplary embodiment is shown and discussed regarding FIG. 9 below.

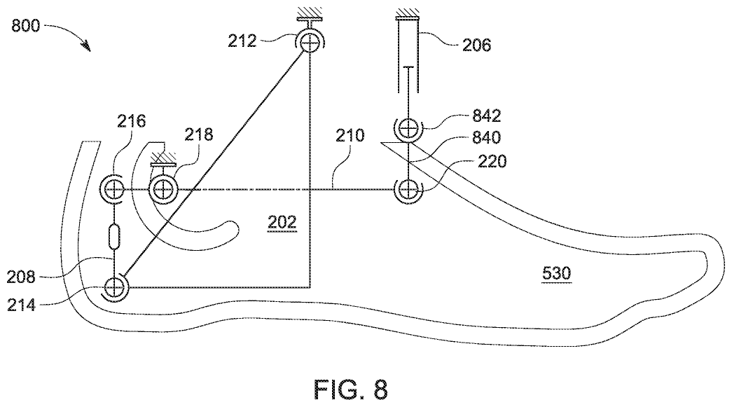

Turning now to FIG. 8, a schematic view of an exemplary ankle joint mechanism 800 with a third coupling link is shown, according to an embodiment of the present disclosure. Similar reference labels in FIG. 8 correspond to elements in FIGS. 2-5 and 6. Ankle joint mechanism 800 further includes a third coupling link 840 and a connection point 842.

The third coupling link 840 is rotatably coupled to the second connecting link 210 at connection point 220 at a first end of the third coupling link 840. A second end, opposite the first end, of the third coupling link 840 is rotatably coupled to the force providing element 206 at connection point 842. Tension and compression for the first connecting link 208 and the force providing element 206 are similar in ankle joint mechanism 800 as in ankle joint mechanism 200. However, when dorsiflexion-inducing loads are applied to ankle joint mechanism 800, the newly introduced coupling link 840 is a two-force member and is under compression (which may lead to buckling). Mechanism 800 advantageously eliminates the sixth pivot point 222 of ankle joint mechanism 200, such that the force providing element 206 is fixed to the shank member 202, and these two components 202, 206 are designed as a single piece, for ease of manufacturing.

FIG. 9 shows a schematic view of an exemplary ankle joint mechanism 900, according to an embodiment of the present disclosure. Similar reference labels in FIG. 9 correspond to elements in FIG. 2-5. In the ankle joint mechanism 900, the second connecting link 210 is able to rotate about the fourth pivot point 218 and the first end of the force providing element 206a is rotatably coupled to the second connecting link 210 at the fifth pivot point 220. Additionally, the fifth pivot point 220 is able to slide relative to the second connecting link 210 in a direction substantially parallel to the second connecting link 210. As the force providing element 206 extends and retracts, the fifth pivot point 210 slides anteriorly and posteriorly along the surface that defines the sliding constraint (shown in FIG. 9 as parallel walls attached to the second connecting link).

While the subject matter of this disclosure has been described and shown in considerable detail with reference to certain illustrative embodiments, including various combinations and sub-combinations of features, those skilled in the art will readily appreciate other embodiments and variations and modifications thereof as encompassed within the scope of the present disclosure. Moreover, the descriptions of such embodiments, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

1. An ankle joint mechanism, comprising:
a shank member comprising a distal portion and a proximal portion;
a first connecting link;
a foot member, wherein the foot member is rotatably coupled to the distal portion of the shank member at a first pivot point, wherein the foot member is rotatably coupled to the first connecting link at a second pivot point, and wherein a distance between the first pivot point and the proximal portion of the shank member is less than a distance between the second pivot point and the proximal portion of the shank member;
a second connecting link, wherein the second connecting link is rotatably coupled to the first connecting link at a third pivot point, and wherein the second connecting link is rotatably coupled to the shank member at a fourth pivot point; and
a linear force providing element, wherein the linear force providing element comprises a first end and second end, wherein the first end of the linear force providing element is coupled to the second connecting link, and wherein the second end of the linear force providing element is coupled to one of the shank member or the foot member.

2. The ankle joint mechanism of claim 1, wherein at least one of a distance from the proximal portion of the shank member to the third pivot point and a distance from the proximal portion of the shank member to the fourth pivot point is greater than a distance from the proximal portion of the shank member to the first pivot point.

3. The ankle joint mechanism of claim 1, wherein a distance from the proximal portion of the shank member to the second pivot point is greater than a distance from the proximal portion of the shank member to the fourth pivot point.

4. The ankle joint mechanism of claim 1, wherein the fourth pivot point is located between the first pivot point and the second pivot point.

5. The ankle joint mechanism of claim 1, wherein at least one of the first connecting link and the second connecting link are configured to be in tension when the ankle joint mechanism is under a dorsiflexive-inducing load.

6. The ankle joint mechanism of claim 1, wherein the linear force providing element is configured to be subject to a compressive force under a dorsiflexive-inducing load.

7. The ankle joint mechanism of claim 1, wherein the first end of the linear force providing element is rotatably coupled to the second connecting link at a fifth pivot point, and wherein the second end of the linear force providing element is rotatably coupled to one of the shank member and the foot member at a sixth pivot point.

8. The ankle joint mechanism of claim 7, wherein the fourth pivot point is positioned on the second connecting link between the third pivot point and the fifth pivot point.

9. The ankle joint mechanism of claim 1, where one of the fourth pivot point, the first end of the linear actuator, or the second end of the linear force providing element comprises a joint configured to rotate and to slide in a linear movement.

10. (canceled)

11. The ankle joint mechanism of claim 1, wherein the linear force providing element comprises:
a first volume of working fluid;
a second volume of working fluid;
a piston;
a valve, wherein the valve connects the first volume of working fluid to the second volume of working fluid on opposing sides of the piston, and wherein the valve is configured to adjust a fluid flow between the first volume of working fluid and the second volume of working fluid; and
at least one sensor configured to vary a shape of the valve between at least two shapes, wherein a first shape of the valve increases the fluid flow and wherein a second shape of the valve decreases the fluid flow.

12. An ankle prosthesis, comprising:
a shank member comprising a distal portion and a proximal portion;
a first connecting link;
a foot member, wherein the foot member is rotatably coupled to the distal portion of the shank member at a first pivot point, wherein the foot member is rotatably coupled to the first connecting link at a second pivot point, and wherein a distance between the first pivot point and the proximal portion of the shank member is less than a distance between the second pivot point and the proximal portion of the shank member;
a second connecting link, wherein the second connecting link is rotatably coupled to the first connecting link at a third pivot point, and wherein the second connecting link is rotatably coupled to the shank member at a fourth pivot point; and
a linear force providing element, wherein the linear force providing element comprises a first end and second end, wherein the first end of the linear force providing element is coupled to the second connecting link, and wherein the second end of the linear force providing element is coupled to one of the shank member or the foot member.

13. The ankle prosthesis of claim 12, further comprising a foot cover, and wherein each of the second pivot point, the third pivot point, and the fourth pivot point are positioned within the foot cover.

14. The ankle prosthesis of claim 12, wherein at least one of the first connecting link, the second connecting link, and the linear force providing element is configured to measure torque applied to the prosthesis.

15. An ankle orthosis, comprising:
a shank member comprising a distal portion and a proximal portion;
a first connecting link;
a foot member, wherein the foot member is rotatably coupled to the distal portion of the shank member at a first pivot point, wherein the foot member is rotatably coupled to the first connecting link at a second pivot point, and wherein a distance between the first pivot point and the proximal portion of the shank member is less than a distance between the second pivot point and the proximal portion of the shank member;
a second connecting link, wherein the second connecting link is rotatably coupled to the first connecting link at a third pivot point, and wherein the second connecting link is rotatably coupled to the shank member at a fourth pivot point; and
a linear force providing element, wherein the linear force providing element comprises a first end and second end, wherein the first end of the linear force providing element is coupled to the second connecting link, and wherein the second end of the linear force providing element is coupled to one of the shank member or the foot member.
